(19) United States
(12) Patent Application Publication
(10) Pub. No.: US 2010/0234722 A1
(43) Pub. Date: Sep. 16, 2010

(54) INTERACTIVE MRI SYSTEM

(76) Inventors: Milan Trcka, Northridge, CA (US); Robert J. Heller, Santa Monica, CA (US)

Correspondence Address: SHELDON MAK ROSE & ANDERSON PC, 100 Corson Street, Third Floor, PASADENA, CA, 91103-3842, US

(21) Appl. No.: 12/487,573

(22) Filed: Jun. 18, 2009

Related U.S. Application Data
(60) Provisional application No. 61/160,128, filed on Mar. 13, 2009.

(51) Int. Cl. A61B 5/055

(52) U.S. Cl. 600/410

(57) ABSTRACT

An MRI system for administering MRI to subjects, and for also providing video and sound to the subjects, and for receiving responses from the subjects. The system includes a sound suppression circuit for suppressing sound emanating from an MRI device. A preferred visual display for use by a subject comprises left and right displays and distance adjusting means for adjusting the distance between the left and right displays. Also preferably comprising LED for receiving video input and transmitting video images through a prism optics to a subject, and a second adjusted means for adjusting the distance between the prism and the LED.

### CROSS-REFERENCE

This application claims the benefit of U.S. Provisional Application No. 61/160,128 filed on Mar. 13, 2009, which is incorporated herein by reference.

### BACKGROUND

Magnetic resonance imaging (“MRI”) systems and functional magnetic resonance imaging (“fMRI”) systems are widely used for diagnosing the physical condition of subjects. They are also used as a research tool for determining the effect of various stimuli on brain activity. For research purposes, it is desirable that audio and/or video stimuli can be provided to a subject undergoing MRI. It is desirable to distract a subject from the MRI process, which can be claustrophobic due to tightly wound head coils. Thus, for even routine MRI, it is desirable that audio and/or visual stimuli be provided. MRI systems that can provide such stimuli are known. See, for example U.S. Pat. No. 5,877,732.

However, existing systems that can provide stimuli suffer from one or more deficiencies, such as inability to be used with high power MRI systems such as those operating at 7 Tesla, discomfort for the subject, and limited capability of the interface system in providing input to the subject and receiving output from the subject.

Accordingly, there is a need for an interactive MRI system that overcomes one or more of these deficiencies of existing systems.

### SUMMARY

An MRI system for administering MRI to subjects comprises an MRI device for use in an MRI room, a control room external to the MRI room, and a shielded interface unit in the MRI room. There is a sound transmission system providing sound to the subject in the MRI. The interface unit receives audio input and has a sound suppression circuit for suppressing sound emanating from the MRI device by generating a sound suppression signal. A sound transmission system provides sound to the subject in the MRI room, wherein the sound transmission system receives the audio input and the sound suppression signal from the interface unit.

A visual display for use by the subject comprises left and right displays and distance adjusting means for adjusting the distance between the left and right displays. Each display can comprise an OLED or other LED system for receiving video input in transmitting video images, and a prism receiving the video images from the OLED or other LED system for viewing by the subject.

### DRAWINGS

These and other features, aspects, and advantages of the present invention will become better understood with reference to the following description, accompanying claims, and appended drawings wherein:

FIG. 1 is a block diagram showing components of a system having features of the present invention;

FIG. 2 is a block diagram of an interface unit for use in the system of FIG. 1;

FIG. 3 is a top down schematic view of the interface unit of FIG. 2;

FIG. 4 is a side schematic view of the interface unit of FIG. 2;

FIG. 5 is a block diagram of an audio/video interface of the interface unit of FIG. 2;

FIG. 6 is a schematic view of an audio/video display device for use with the system of FIG. 1;

FIG. 7 is a perspective view of an audio/video device useful in the system of FIG. 1;

FIG. 8 shows an audio video device of FIG. 7 on a subject;

FIG. 9 is a wiring diagram of the audio/video device;

FIG. 10A is a perspective view of a video display for use in the system of FIG. 1;

FIG. 10B is a side elevation view of the display of FIG. 10A;

FIG. 10C schematically shows the video display of FIG. 10A;

FIG. 11 shows a speaker system for use in the system of FIG. 1;

FIG. 12 shows a microphone for use in the system of FIG. 1;

FIG. 13A is a top plan schematic view of a call button for use in the system of FIG. 1;

FIG. 13B is a front schematic view of the call button of FIG. 13A; and

FIG. 13C shows components of the call box system of FIG. 13A.

### DESCRIPTION

### Introduction

According to one embodiment of the present invention, there is provided a device for performing interaction with a subject in Magnetic Resonance Imaging (MRI) or functional Magnetic Resonance Imaging (fMRI) devices. According to another embodiment of the present invention, there is provided a system for performing MRI or fMRI on a subject. According to another embodiment of the present invention, there is provided a method for performing MRI or fMRI on a subject. In one embodiment, the method comprises providing a device according to the present invention and using the device to perform magnetic resonance imaging on a subject.

As used herein, except where the context requires otherwise, the term “comprise” and variations of the term, such as “comprising,” “comprises” and “comprised” are not intended to exclude other additives, components, integers or steps. Thus, throughout this specification, unless the context requires otherwise, the words “comprise”, “comprising” and the like, are to be construed in an inclusive sense as opposed to an exclusive sense, that is to say, in the sense of “including, but not limited to”.

As used herein the terms fMRI-compatible and MRI-compatible refer to devices that are intended for use during fMRI and MRI procedures, respectively, such that neither the data recorded by the device nor the data recorded by the procedure are reasonably considered as detrimentally affected by the joint usage of fMRI or MRI in practice.

An MRI-compatible device does not guarantee fMRI-compatibility. Examples of methods to make devices fMRI-compatible include, but are not limited to, use of non-ferromagnetic materials, such as plastic, to reduce attractive forces between the device and the superconducting magnet of the MRI scanner, and shielding to reduce electromagnetic interference that could corrupt the data measured device and corrupt the signal-to-noise ratio or contrast-to-noise ratio of the data.

As depicted in the Figures, all dimensions specified in this disclosure are by way of example only and are not intended to be limiting. Further, the proportions shown in these Figures are not necessarily to scale. As will be understood by those with skill in the art with reference to this disclosure, the actual dimensions of any device or part of a device disclosed in this disclosure will be determined by its intended use.

### Overview of the System

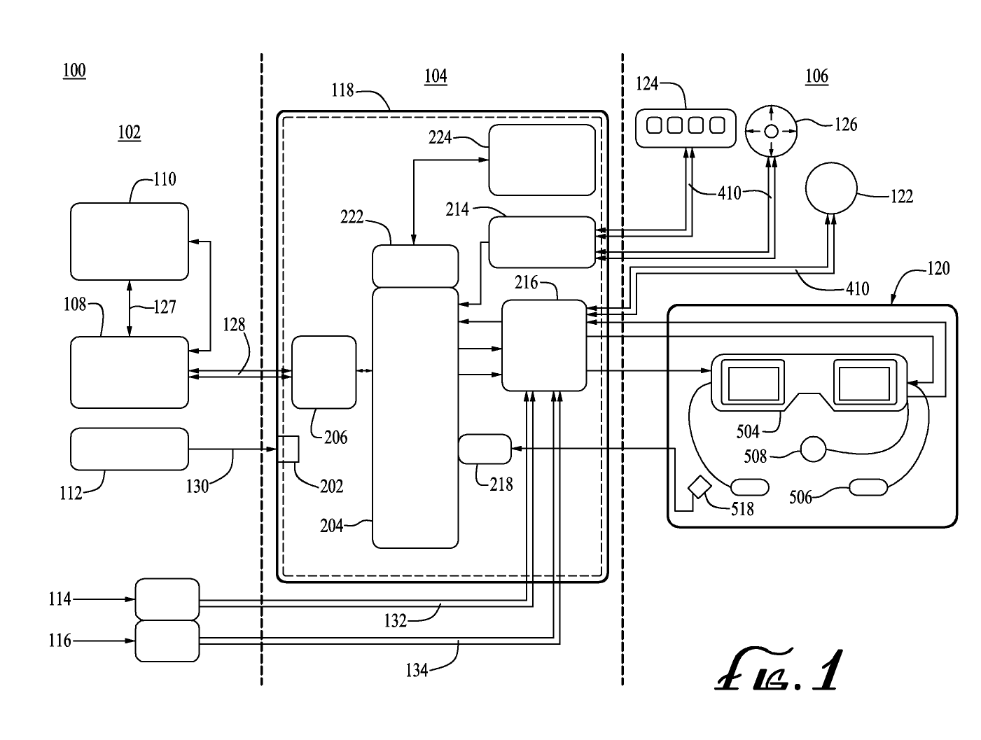

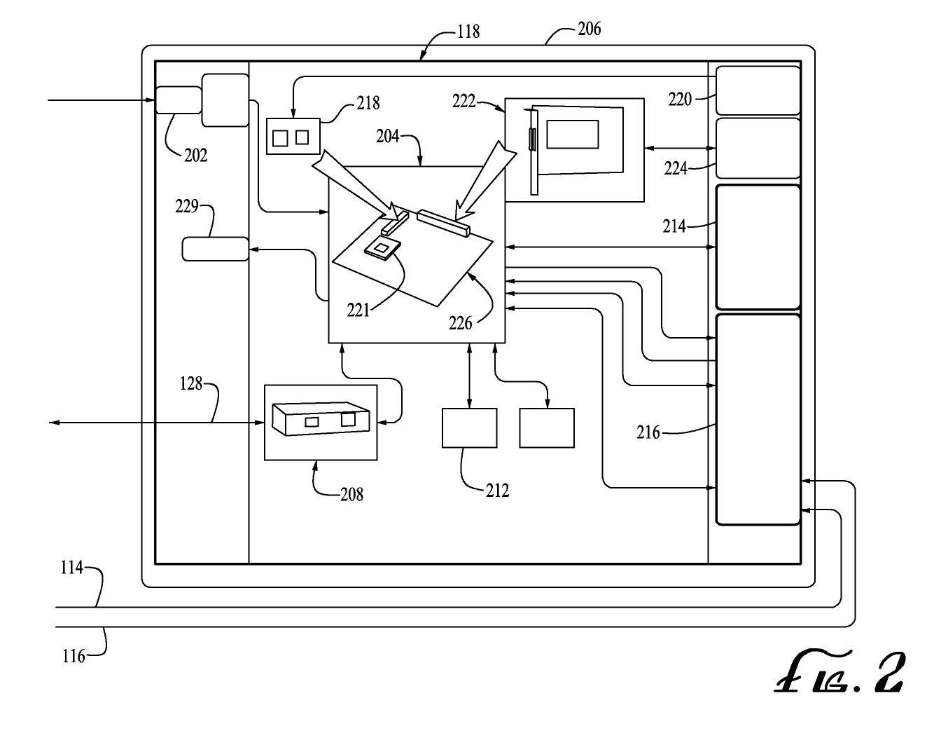

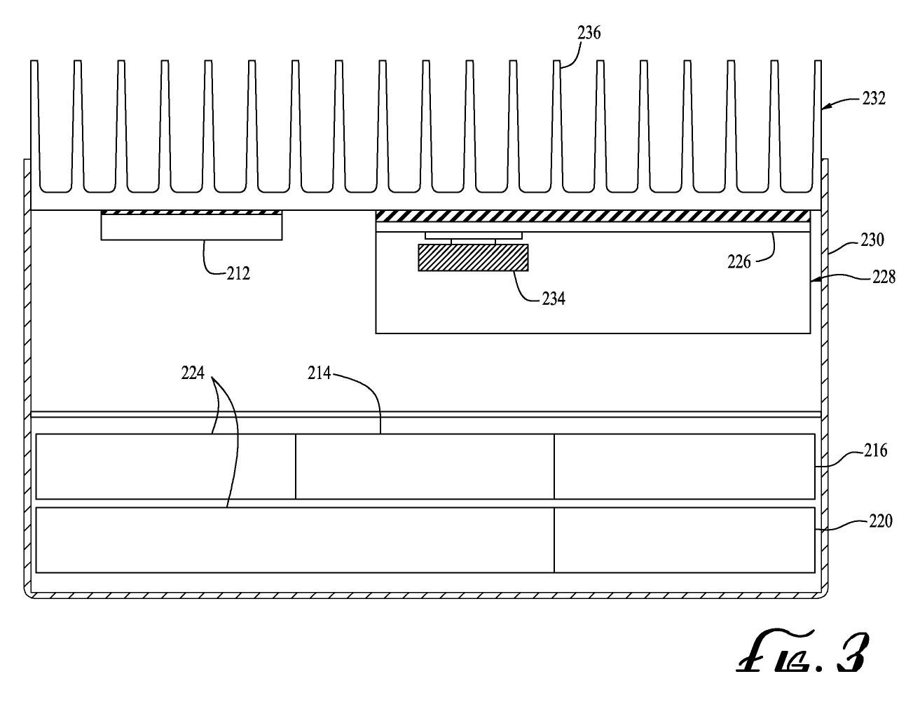

Referring now to FIG. 1, there is shown a system 100 having features of this invention. The system 100 comprises a control room 102 and an MRI room 104, wherein the MRI room comprises a magnet bore 106. The term “MRI room” also includes a room used for fMRI.

The control room 102 comprises a computer work station 108 optionally operated via a touch display screen 110 for controlling the system 100, a power supply 112, a video feed 114 providing video input, and an audio feed 116 providing audio input. The video feed 114 and the audio feed 116 can be optionally connected to the computer work station 108 or to any other device capable of video or audio output such as a DVD player (not shown). The MRI room 104 comprises an interface unit 118.

Within the magnet bore 106 are subject interface devices such as a portable audio visual system 120, a call button 122, a response device 124, and a manual controller such as a joystick 126, all of which are connected to the interface unit 118.

### Control Room Components

The computer work station 108 can be any conventional computer such as those provided by Dell®, Hewlett-Packard®, and others. It typically includes computer memory, USB ports, a printer, a monitor, a keyboard, and a mouse. Optionally the monitor can be in the form of the touch display screen 110. The display touch screen 110 can be connected to the PC work station 108 through a standard USB connector 127. The work station 108 communicates with the interface unit 118 through an optical communication line 128. The power supply 112 provides external power to a power line 130 to the interface unit 118, and through the interface unit 118 to the magnet bore components. The power can be 12 volt DC. The optical connection line 128 is used for providing control signals and other input to the interface unit 118 and for receiving input obtained from a subject to the work station 108 to the control room. Preferably a single cable is used for transmitting the power and the control signals to the MRI room and for transmitting input from the subjects to the control room 102.

The video feed 114 and the audio feed 116 are transmitted to the interface unit 118 through respective SVGA fiber optic lines 132 and 134.

### Interface Unit

Referring to FIGS. 1 and 4, the interface unit 118 comprises a transformer 202 connected to the power supply 112, an interface computer 204 connected to the transformer 220, a magnetic shielding housing 206 surrounding the interface computer 204, a network interface 208 connected electronically to the interface computer 204 and connected to the computer workstation 108 via the optical Ethernet connection 128 using optical signals, a data storage unit 212 connected electronically to the interface computer 204, an auxiliary interface 214 connected electronically to the interface computer 204, and an audio/video goggle interface 216 connected electronically to the interface computer 204, wherein the interface unit 118 is sufficiently shielded by the magnetic shielding housing 206 that it can be used in the MRI room 104 exported to the MRI magnetic field. The interface unit 118 optionally comprises a video capture card 218 connected to the interface computer 204, an optional eye tracker interface 220 connected electronically to the video capture card 218, a data acquisition unit 222 connected electronically to the interface computer 204, and an optional subject monitor receiver 224 connected electronically to the data acquisition unit 222.

The interface computer 204 comprises a circuit board 226 and a single board computer (SBC) 228. In a preferred embodiment, the circuit board 226 is a printed circuit board and the SBC 228 is a mini-ITX motherboard, such as a Commell™ LV-679 available from Taiwan Commate Computer, Inc., 8F, No. 94, Sec. 1, Shin Tai Wu Rd., Hsin Chin, Taipei Hsien, Taiwan. In a preferred embodiment, the interface computer 204 is loaded with active noise cancellation (ANC) software such as described in U.S. Pat. No. 5,427,102 or U.S. Pat. No. 5,440,641. This software enables a background audio input into the interface computer, software to produce an output sound that is a 180-degree phase-shift sound from the background audio input such that the output sound cancels the background audio input, and an audio output to deliver the output sound. In this way the subject benefits by not having the typical MRI noise around. The administrator benefits through better research results, as the subject is less likely to move around. In another preferred embodiment, the SBC 228 further comprises a DVI monitor output 229 for outputting video image to the computer work station 108.

The magnetic shielding housing 206 can comprise a computer housing 230 containing the interface unit 118 and a cooler 232 thermally connected to the interface computer 204 and to the data storage unit 212, which has a heat sink 236. In a preferred embodiment, the cooler 232 comprises a high surface area grid to conduct heat to surrounding air. In an especially preferred embodiment, the high surface area grid is comprised of aluminum and has dimensions 12.25″×2.3″×12″. In a preferred embodiment, the cooler 232 is thermally connected to the circuit board 226 by means of a thermally conductive gap filler, such as Berquist™ GP2500S20, available from The Berquist Company in Chanhassen, Minn., measuring 6.7″×6.7″×0.2″. In a preferred embodiment, the interface computer 204 further comprises air and the cooler 232 further comprises a CPU cooler 234 in thermal contact with the circuit board 226, the air inside the interface computer 204, and the heat sink 236. With this design, there is significant cooling with a few moving parts that can interfere with the MRI or fMRI.

The network interface 204 is a converter box capable of converting the optical signal into a standard electronic signal for use in the interface computer 204. In a preferred embodiment, the network interface 204 is a Copper Gigabit Ethernet to Small Form-factor Pluggables (SFP) Fan-less system and is connected to the interface computer 204 with a 1000-BaseT Ethernet connection and to the computer workstation 108 by a 1000-Base SX Gigabit Optical Ethernet cord. The Copper Gigabit Ethernet to SFP Fan-less system can be an Allied Telesis® AT-MC1008/SP 100T available from Allied Telesis, 19800 North Creek Parkway, Bothell Wash. The 1000-Base SX Gigabit Optical Ethernet cord can be an Opticis North America® CAB-DVIFO-30MM available from 330 Richmond St., Chatham, Ontario, Canada.

In a preferred embodiment, the data storage unit 212 is a solid-state hard drive without moving parts and is connected to the heat sink 236. The solid-state hard drive can be an Intel® SSD 80 GB storage unit, available from Intel Corporation, 2200 Mission College Blvd, Santa Clara, Calif. The data storage unit 212 is connected to the interface computer 204 by a serial ATA (SATA) connection. In another preferred embodiment, there is a plurality of data storage units, each connected to the interface computer 204 and to the heat sink 236. Solid-state hard drives are better for use in the MRI room due to their lack of moving parts. Typical hard drives have electric motors that can interfere with MRI and fMRI.

In a preferred embodiment, the auxiliary interface 214 is connected to the interface computer 204 through a standard two-way electronic communication means, such as a USB cable or wirelessly. The auxiliary interface 214 comprises a circuit to convert between electrical and optical signals and communication means to send and receive an optical signal through fiber optic cables. An example of the communication means is a photodiode circuit, light-emitting diode (LED), or photodetector, such as an Industrial Fiber Optics® IF-E96 for converting electrical signals into optical signals and an Industrial Fiber Optics® IF-D95 for converting optical signals into electrical signals, both available from Industrial Fiber Optics, Inc., 1725 West 1st Street, Tempe, Ariz.

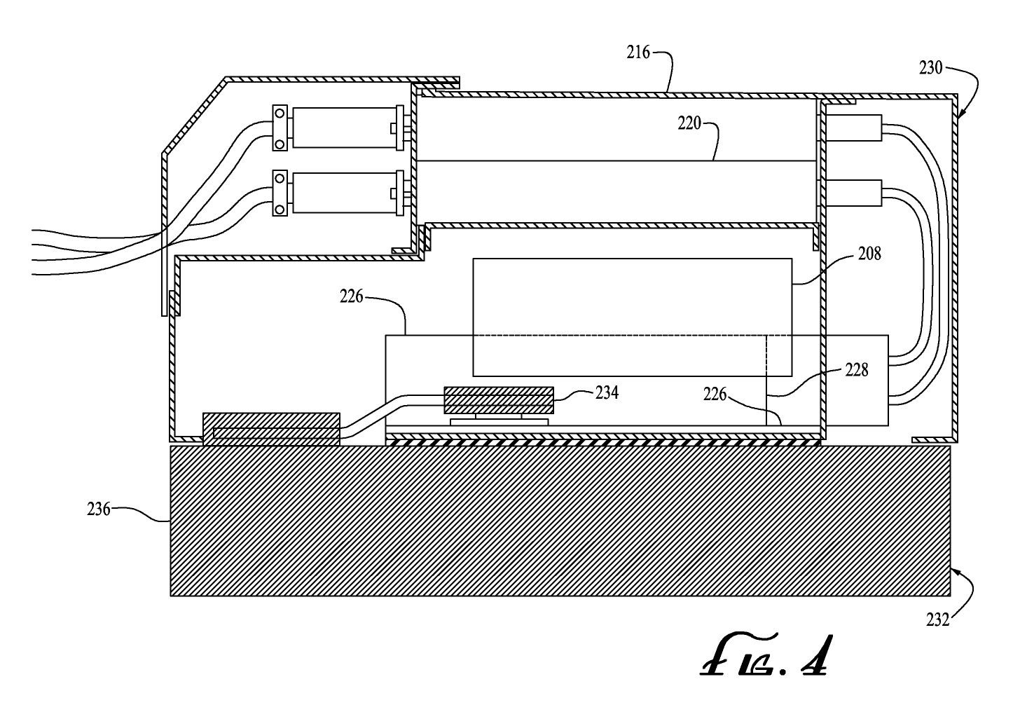

The interface unit 118 includes an audio/video goggle interface 216, shown in FIG. 5. The audio/video goggle interface 216 comprises a non-magnetic male electrical connector 302 connected to the SBC 228; a Digital Visual Interface (DVI) connector 304 connected to the control room 102; a front panel (FP) audio connector 306 connected to the control room 102; an interface system 308 connected to the DVI connector 304, to the FP audio connector 306, and to the non-magnetic male electrical connector 302; a non-magnetic female electrical connector 310 electrically connected to the interface system 308; and a fiber connector 312 electrically connected to the interface system 308. In a preferred embodiment, the non-magnetic male electrical connector 302 and the non-magnetic female electrical connector 310 are ITT Cannon® D-Subminiature non-magnetic connectors available from ITT Interconnect Solutions, 5288 Valley Industrial Blvd S, Shakopee, Minn.

The DVI connector 304 is configured to receive video information from the video feed 114, while the FP audio connector 306 is configured to receive audio information from the audio feed 116. The non-magnetic male electrical connector 302 is configured to have a plurality of electrical connections with the SBC, including an SBC video signal connection 314, an SBC communication signal connection 316, an SBC audio signal connection 318, a microphone SBC connection 320, and a power connection 322.

The interface system 308 comprises a DVI to Super Video Graphics Array (SVGA) converter 324 connected to the DVI connector 304 through a DVI cable 305; a video selector 326 connected to the DVI to SVGA converter 324 through an SVGA cable and connected to the SBC video signal connection 314 through a cable communicating SVGA, color, and synchronicity video information; an interface controller 328 connected to the SBC communication signal connection 316 by a two-way connection cable, such as a USB cable; a control logic 330 connected to the interface controller 328; a digital to analog converter (DAC) 332 connected to the control logic 330 with an interface such as a two-wire interface (TWI) or serial peripheral interface (SPI); a fiber receiver DAC 334 connected to the FP audio connector 306 by an optical cable and configured to convert an optical audio signal to an electric signal; an audio mixer 336 connected to the fiber receiver DAC 334 and to the SBC audio signal connection 318, configured to combine the two audio signals into one electrical signal; a speaker amp 338 connected to the audio mixer 336; a communication microphone line amplifier 340 connected to the microphone SBC connection 320; a regulator 342 connected to the power connection 322; a first electro-optical converter 344 connected to the control logic 330 and configured to convert an electrical signal to an optical signal; and a second electro-optical converter 346 connected to the control logic 330 and configured to convert an optical signal into an electrical signal. In a preferred embodiment, the first electro-optical converter 344 is an Industrial Fiber Optics® IF-E96 and the second electro-optical converter 346 is an Industrial Fiber Optics® IF-D95, both available from Industrial Fiber Optics, Inc., 1725 West 1st Street, Tempe, Ariz.

The non-magnetic female electrical connector 310 is configured to have plurality of electrical connections with the audio/video goggle interface 216, such as a display drive 348 connected to the video selector 326 through a video cable such as SVGA to communicate video signal, a display control 350 connected to the control logic 330, a voltage output 352 connected to the DAC 332, a speaker connection 354 connected to the speaker amp 338, a microphone connection 356 connected to the communication line microphone amplifier 340, and a goggle power connection 358 connected to the regulator 342. The fiber connector 312 comprises a call button connector 360 connected to the first electro-optical converter 344 and to the second electro-optical converter 346.

In a preferred embodiment, audio/video goggle interface 216 further comprises a noise cancellation connection 362 in the non-magnetic female electrical connector 310; and a noise canceling microphone interface 364 located in the interface system 308 and connected to the noise cancellation connection 362, to the control logic 330, and to the audio mixer 336 in such a way as to deliver background noise for active noise cancellation from the interface computer 204 in an audio output.

In a preferred embodiment, the video capture card 218 is a Commell® mini-PCI, available from Taiwan Commate Computer, Inc., 8F, No. 94, Sec. 1, Shin Tai Wu Rd., Hsin Chin, Taipei Hsien, Taiwan, and is connected to the eye tracker interface 220 through a NTSC Video cable.

The eye tracker interface 220 is capable of receiving video image from a fiber optic cable and converting the signal from the fiber optic cable into an electrical signal.

In a preferred embodiment, the data acquisition unit 222 is a 16 channel National Instruments® DAQ NI PCIe-6259, available from National Instruments Corp., 11500 N Mopac Expwy., Austin, Tex., and is connected to the interface computer 204 through a Peripheral Component Interconnect Express (PCIe) connection. In a preferred embodiment, the data acquisition unit 222 is configured to receive both digital and analog electrical signals from the subject monitor receiver 224.

In a preferred embodiment, the subject monitor receiver 224 is capable of receiving signals from the subject regarding the subject's heart rate, respiration, temperature, oxygen levels, and brain electrical activity according to methods known in the art, such as U.S. Pat. No. 6,731,976, and U.S. Pat. No. 6,533,733.

### Magnet Bore Components

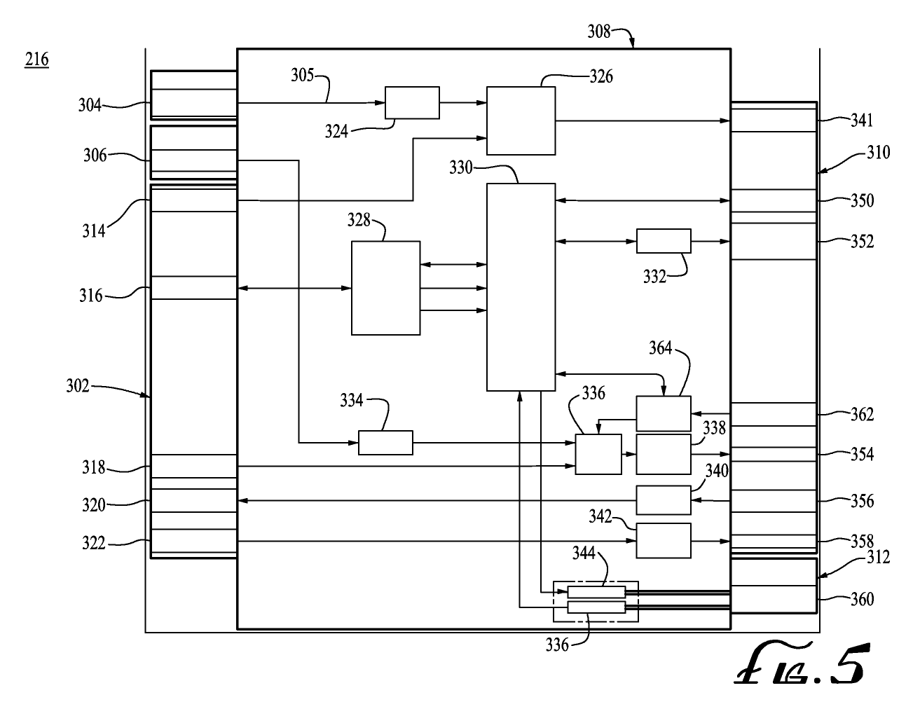

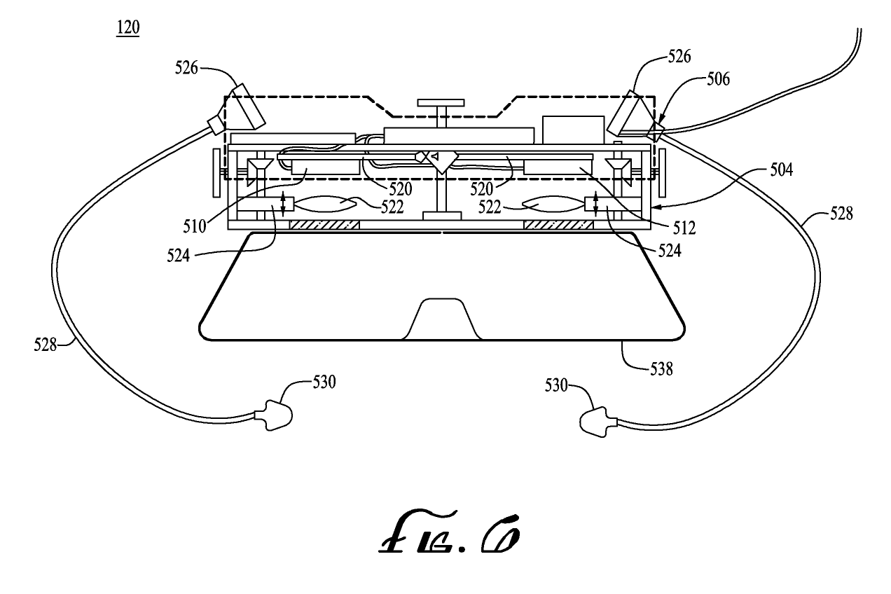

Referring now to FIGS. 6-9, items in the magnet bore 106 can be seen.

The audio/video goggle system 120 is connected to the audio/video goggle interface 216 through an electronic cable having a second non-magnetic male connector 502 with a ground connection 503 and comprises a visual display 504, a sound transmission system 506 connected to the second non-magnetic male connector 502 through audio cables 507, and a microphone system 508. The second non-magnetic male connector 502 connects to the non-magnetic female connector 310. The visual display 504 is connected to the audio/video goggle interface 216 through cables communicating video information 509 and comprises a left display 510; a right display 512; a display logic 514 connected to the second non-magnetic male connector 502 through logic cables 515, to the left display 510, and to the right display 512; and a plurality of voltage controllers 516 connected to the second non-magnetic male connector 502 through voltage cables 517, to the left display 510, and to the right display 512. The audio/video goggle system 120 optionally further comprises an eye tracker system 518 that is connected to the optional eye tracker interface 220. The microphone system 508 is connected to the second non-magnetic male connector 502 through a microphone cable 519.

In a preferred embodiment, the left display 510 and right display 512 each further comprise an organic light-emitting diode (OLED) system or other LED system 520 for receiving and transmitting video images, a prism or mirror system 522 for receiving video images from the OLED system or LED system 520, and a diopter adjustment mechanism 524 for adjusting the distance between the prism or mirror system 522 and the OLED system or LED system 520. The diopter adjustment mechanism 524 can be manual, such as a threaded rod, or can comprise a non-magnetic motor, such as a miniature piezoelectric micromotor, such as a Squiggle® motor. The prism or mirror system 522 receives the video signal from the OLED system or LED system 520 and transmits it to the subject without the need for a lens. The OLED system or LED system 520 and the prism or mirror system 522 used can be an eMagin® WF05 optics module, comprising an active matrix OLED-on-Silicon microdisplay, available from eMagin Corporation, 10500 NE 8th Street, Bellevue, Wash. This module is the preferred display mechanism since its display does not degrade in magnetic fields up to at least 7 Tesla.

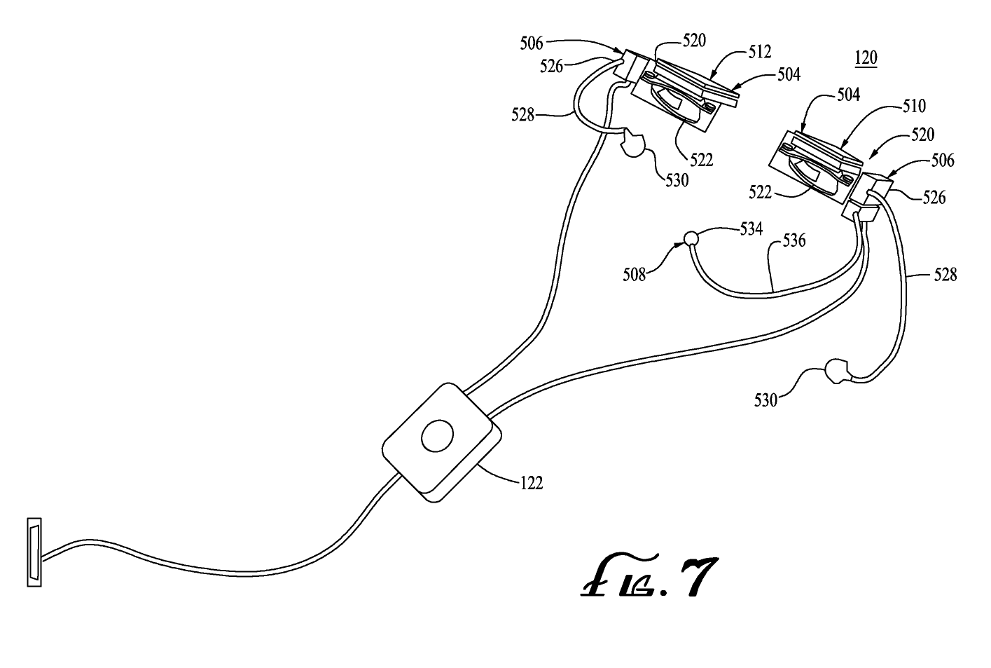

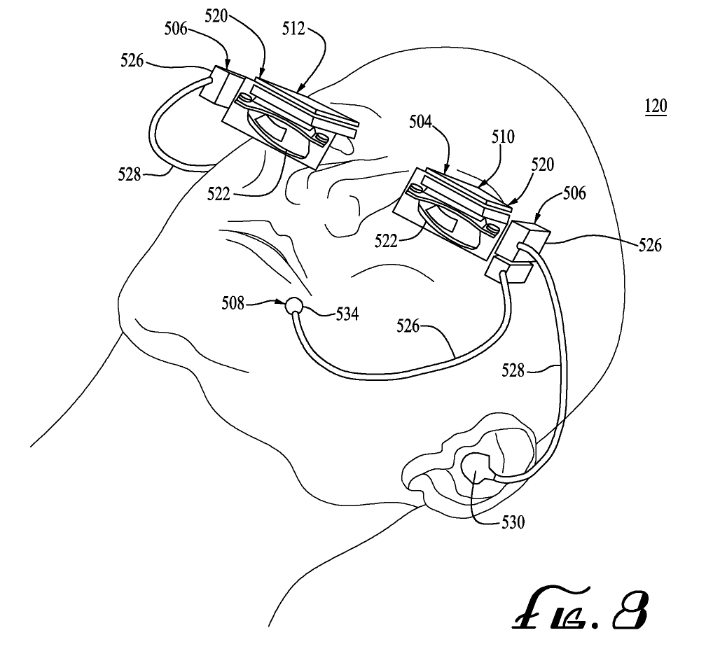

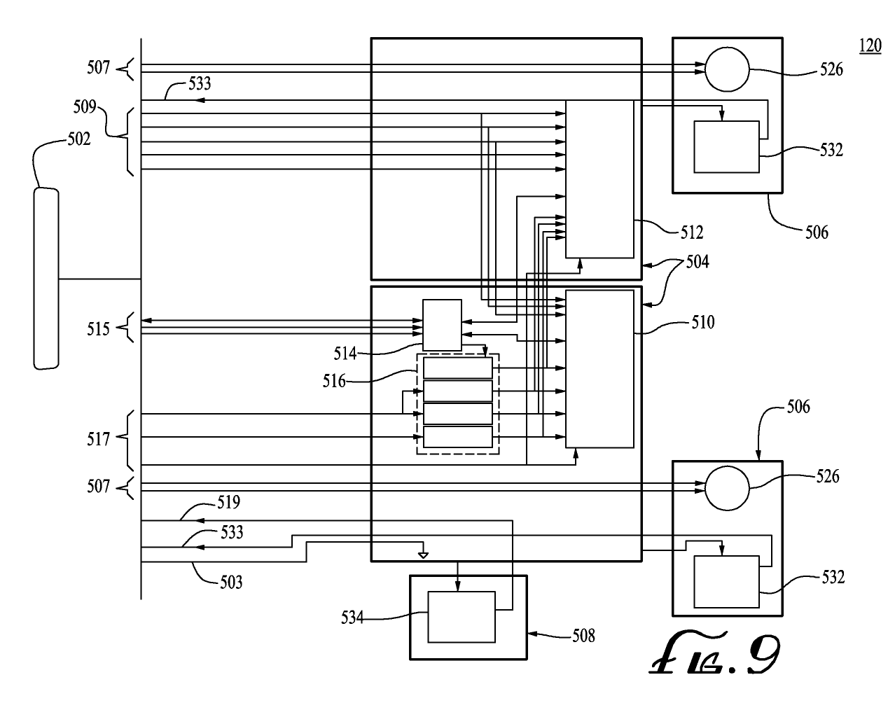

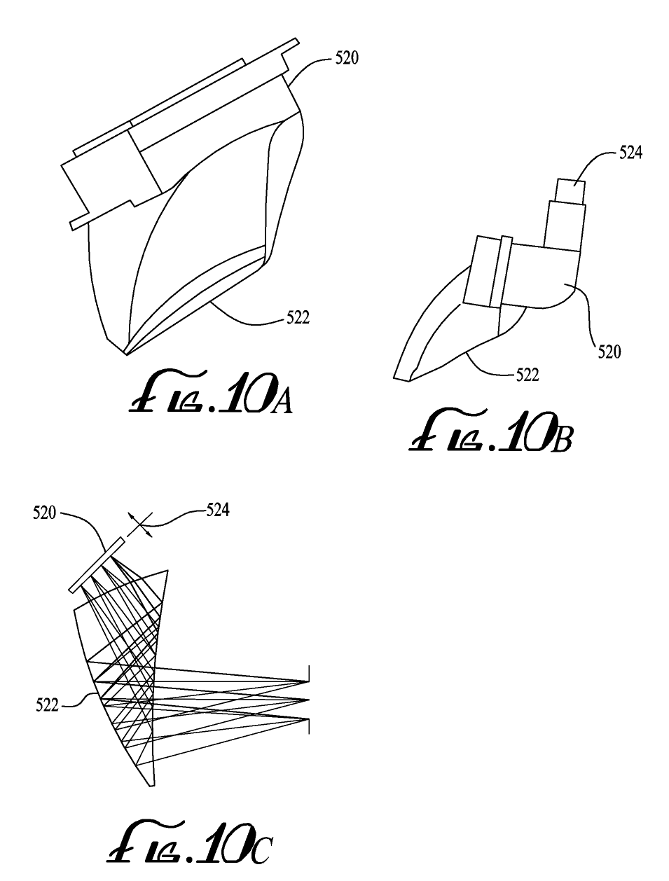

The sound transmission system 506 can especially be seen in FIG. 11. The sound transmission system 506 used is a modified version of a Mallory Sonalert Products® PT-2060WQ, available from Mallory Sonalert Products, Inc., 4411 South High School Road, Indianapolis, Ind., and comprises a piezoelectric speaker 526 that converts an electric signal to an acoustic audio signal, an acoustic waveguide 528 receiving the audio signal and attached to the piezoelectric speaker 526, and an earpiece 530 attached to the acoustic waveguide 528 and located proximal to a subject's ear. The Mallory Sonalert Products® PT-2060WQ is modified through wire-stripping and magnetically shielding with a material capable of magnetic shielding, such as mylar or copper braiding. The sound transmission system 506 can also further comprise ceramic speakers. In a preferred embodiment, the sound transmission system 506 further comprises noise cancellation microphones 532 that pick up MRI background noise. These noise cancellation microphones 532 deliver an audio signal to the audio/video goggle interface 216 through noise cancellation cables 533, shown in FIG. 9.

The microphone system 508 can especially be seen in FIG. 12. The microphone system 508 used is a non-magnetic microelectromechanical system (MEMS) microphone 534 connected to the microprocessor through an acoustic waveguide 536, wherein the acoustic waveguide 536 is configured to have an opening near the subject's mouth for receiving verbal communication. In a preferred embodiment, the MEMS microphone 534 is an analog output single chip MEMS microphone with an integrated transducer and associated circuitry on a single piece of silicon, such as an Akustica® AKU1126, available from Akustica, Inc., 2835 East Carson Street, Suite 301, Pittsburg, Pa., and modified through wire-stripping and magnetically shielding with a material capable of magnetic shielding, such as mylar or copper braiding.

In a preferred embodiment, the visual display 504, sound transmission system 506, and microphone system 508 are a unitary unit having the general shape of binocular goggles, and the audio/video goggle system 120 further comprises an inter-pupillary adjustment mechanism. The inter-pupillary adjustment mechanism can be manual, such as a threaded rod, or comprise a non-magnetic motor. The audio/video goggle system 120 is mounted to a face module made of a bio-compatible non-magnetic material, such as flexible plastic, silicone, or polyurethane. In another preferred embodiment, the audio/video goggle system 120 further comprises a removable shield 538 for placement on the unitary unit between the unitary unit and the subject. In a preferred embodiment, the audio/video goggle system 120 further comprises a strap securing the audio/video goggle system 120 to the subject. In another preferred embodiment, the audio/video goggle system 120 is connected to the audio/video goggle interface 216 through a single 37-pin cable, as shown in FIG. 9. The single cable can also be magnetically shielded through braided shielding as is known in the art and has the advantage of minimizing interference with the MRI or fMRI.

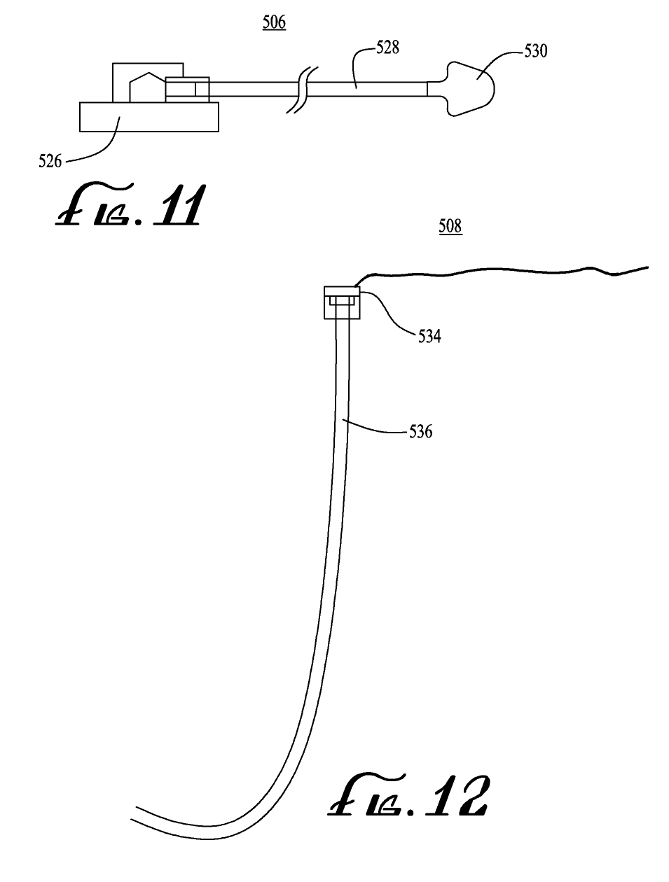

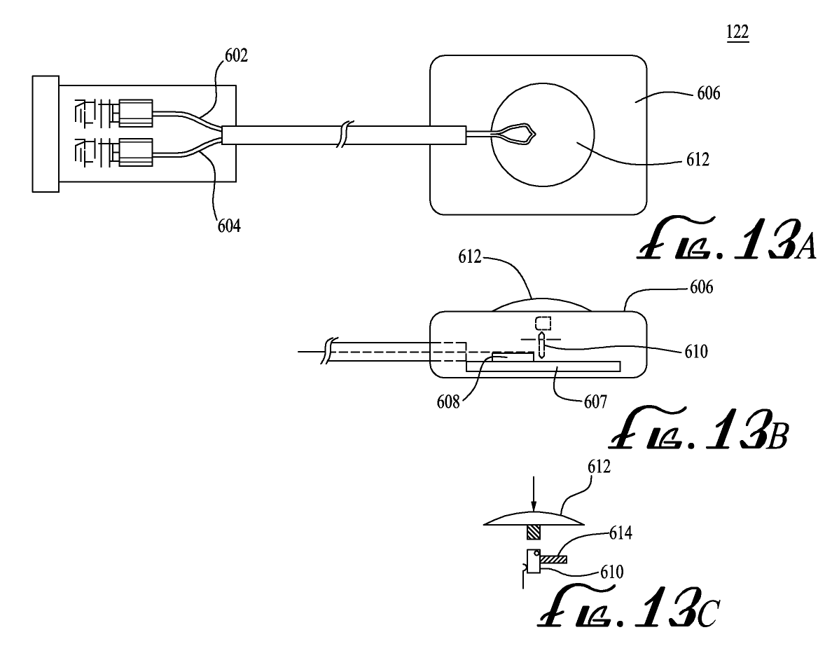

The call button 122 can especially be seen in FIGS. 13A-C. The call button 122 comprises a first fiber optic cable 602 having a first end and an opposed second end, wherein the first end is closer to the subject than to the control room 102; a second fiber optic cable 604 having a first end and an opposed second end, wherein the first end is closer to the subject than to the control room 102; a housing 606 holding the first end of the first fiber optic cable 602 and the first end of the second fiber optic cable 604 in such a way as to make the first end of the first fiber optic cable 602 and the first end of the second fiber optic cable 604 proximal to each other using a base 607 and a fiber support 608 so that there is an optical path between the two fiber optic cables; a light interruption mechanism 610 within the housing 606 such as a mirror or prism that is configured to come between the first end of the first fiber optic cable and the first end of the second fiber optic cable; a disk 612 attached to the light interruption mechanism 610, located outside of the housing 606, and configured in such a way that a subject blocks the optical path by pushing down on the disk; and a spring 614 such that when a subject pushes the disk down the spring 614 delivers a force to push the disk back up and re-open the optical path. The first fiber optic cable 602 and the second fiber optic cable 604 are connected to the call button connector 360 of the audio/video goggle interface 216. The first fiber optic cable 602 receives an optical input from the audio/video goggle interface 216 and transmits it to the second fiber optic cable 604.

The response device 124 comprises at least one input button. Each button is constructed in a similar way to the call button 122 and comprises a first fiber optic cable having a first end and an opposed second end, wherein the first end is closer to the subject than to the control room 102; a second fiber optic cable having a first end and an opposed second end, wherein the first end is closer to the subject than to the control room 102; a housing holding the first end of the first fiber optic cable and the first end of the second fiber optic cable in such a way as to make the first end of the first fiber optic cable and the first end of the second fiber optic cable proximal to each other using a base and a fiber support so that there is an optical path between the two fiber optic cables; a light interruption mechanism within the housing such as a mirror or prism that is configured to come between the first end of the first fiber optic cable and the first end of the second fiber optic cable; a disk attached to the light interruption mechanism, located outside of the housing, and configured in such a way that a subject blocks the optical path by pushing down on the disk; and a spring such that when a subject pushes the disk down the spring delivers a force to push the disk back up and re-open the optical path. The first fiber optic cable and the second fiber optic cable are connected to the auxiliary interface 214. In another embodiment, there is plurality of subject input buttons.

The manual controller, or joystick, 126 is constructed in a similar way to the call button 122 and comprises a first fiber optic cable having a first end and an opposed second end, wherein the first end is closer to the subject than to the control room 102; a second fiber optic cable having a first end and an opposed second end, wherein the first end is closer to the subject than to the control room 102; a housing holding the first end of the first fiber optic cable and the first end of the second fiber optic cable in such a way as to make the first end of the first fiber optic cable and the first end of the second fiber optic cable proximal to each other using a base and a fiber support so that there is an optical path between the two fiber optic cables; a light interruption mechanism within the housing such as a mirror or prism that is configured to come between the first end of the first fiber optic cable and the first end of the second fiber optic cable in an incremental way; a hand-held control stick attached to the light interruption mechanism, located outside of the housing, and configured in such a way that a subject partially blocks the optical path by moving the control stick in a direction; and a spring such that when a subject moves the control stick the spring delivers a force to push the control stick back into a its original position and re-open the optical path. When a subject partially blocks the optical path, an analog signal is sent to the auxiliary interface 214. The first fiber optic cable and the second fiber optic cable are connected to the auxiliary interface 214.

In another embodiment, the joystick 126 comprises a first plurality of fiber optic cables, wherein each fiber optic cable of the first plurality of fiber optic cables has a first end and an opposed second end, wherein the first end of each of the fiber optic cables is closer to the subject than to the control room 102; a second plurality of fiber optic cables, wherein each fiber optic cable of the second plurality has a corresponding fiber optic cable of the first plurality and forms a pair, wherein each fiber optic cable of the second plurality of fiber optic cables has a first end and an opposed second end, wherein the first end of each of the fiber optic cables is closer to the subject than to the control room 102; a housing holding the first end of each of the fiber optic cables in such a way as to make the first end of each fiber optic cable of the second plurality proximal to the first end of each corresponding fiber optic cable of the first plurality using a base and a fiber support so that there is an optical path between each pair of fiber optic cables forming a plurality of optical paths; a light interruption mechanism within the housing such as a mirror or prism that is configured to interrupt the optical path between one or more than one of the fiber optic cable pairs; a hand-held control stick attached to the light interruption mechanism, located outside of the housing, and configured in such a way that a subject blocks one or more than one optical path by moving the control stick in a direction; and a spring such that when a subject moves the control stick the spring delivers a force to push the control stick back into a its original position and re-open the optical paths. The first plurality of fiber optic cables and the second plurality of fiber optic cables are connected to the auxiliary interface 214.

In another embodiment, there is an audio adjustment mechanism, comprising a first plurality of fiber optic cables, wherein each fiber optic cable of the first plurality of fiber optic cables has a first end and an opposed second end, wherein the first end of each of the fiber optic cables is closer to the subject than to the control room 102; a second plurality of fiber optic cables, wherein each fiber optic cable of the second plurality has a corresponding fiber optic cable of the first plurality and forms a pair, wherein each fiber optic cable of the second plurality of fiber optic cables has a first end and an opposed second end, wherein the first end of each of the fiber optic cables is closer to the subject than to the control room 102; a housing holding the first end of each of the fiber optic cables in such a way as to make the first end of each fiber optic cable of the second plurality proximal to the first end of each corresponding fiber optic cable of the first plurality using a base and a fiber support so that there is an optical path between each pair of fiber optic cables forming a plurality of optical paths; a light interruption mechanism within the housing such as a mirror or prism that is configured to interrupt the optical path between one or more than one of the fiber optic cable pairs; a knob attached to the light interruption mechanism, located outside of the housing, and configured in such a way that a subject blocks one or more than one optical path by moving the knob in a direction; and a spring such that when a subject moves the knob the spring delivers a force to push the knob back into a its original position and re-open the optical paths. The first plurality of fiber optic cables and the second plurality of fiber optic cables are connected to the auxiliary interface 214. The audio adjustment mechanism is configured in such a way to adjust audio properties of the audio signal in the earpiece 530.

Although the present invention has been discussed in considerable detail with reference to certain preferred embodiments, other embodiments are possible. Therefore, the scope of the appended claims should not be limited to the description of preferred embodiments contained in this disclosure.

### Advantages

The previously described embodiments of the present invention have many advantages, including an audio/video system with minimal magnetically susceptible components and a compact design for fitting into tighter head coils.

### Features of the System

The following summarizes certain features of the system:

1. A system for use in an MRI device used with a subject comprising:

a) an interface comprising a microprocessor for receiving a video input and an audio input, and for receiving subject generated sound input and subject generated control input;

b) a visual display for receiving from the interface the video input and for displaying to the subject visual images, the video display comprising left and right displays and first adjustment means for adjusting the distance between the left and right displays, each display comprising:
i) an OLED for receiving the video input and transmitting video images;
ii) a prism receiving the video images from the OLED; and
iii) second adjustment means for adjusting the distance between the prisms and the OLED;

c) a sound suppression circuit in the interface for suppressing sound emanating from the MRI device by generating a sound suppression signal;

d) a sound transmission system wearable by the subject, wherein the sound transmission system receives the audio input and the sound suppression signal from the interface;

e) a microphone system for receiving subject generated sound for transmission to the interface as subject generated sound input; and

f) a subject controllable input device for providing subject inputs to the interface; and

g) a subject monitor receiver in the interface for receiving physiological information about a subject.

wherein the system is sufficiently shielded that it can be used in an MRI room.

2. The system of claim 1 wherein the visual display, the sound transmission system, and the microphone system are a single subject wearable unit having the general shape of binocular goggles.

3. The system of claim 1 comprising a protective removable shield between the subject wearable unit and the subject.

4. The system of claim 1 wherein the first adjustment means comprises a motor.

5. The system of claim 1 wherein the second adjustment means comprises a motor.

6. The system of claim 1 wherein the subject controllable input device comprises a button, light input guide, light output guide, and a mirror reflecting input light from the light input guide to the light output guide.

7. The system of claim 2 wherein the sound transmission system comprises a pair of ceramic speakers proximate to the visual display, a sound transmitting flexible tube from each speaker to a respective ear bud.

8. The system of claim 2 wherein the microphone system comprises a microphone proximate to the visual display and a flexible tube sufficiently long to be proximate to a subject's mouth.

9. An MRI system for administering MRI to subjects, the MRI system comprising:

a) an MRI device for use in an MRI room;

b) a control room external to the MRI room;

c) an interface in the MRI room for receiving control signals and audio and video inputs and power from the control room, and for transmitting input from subjects to the control room; and

d) a single cable for transmitting the power and the control signals to the MRI room, and for transmitting input from subjects to the control room.

10. A visual display for use by a subject in an MRI, the video display comprising left and right displays and distance adjustment means for adjusting the distance between the left and right displays, each display comprising:
i) an OLED for receiving the video input and transmitting video images; and
ii) a prism receiving the video images from the OLED.

11. The system of claim 10 wherein the adjustment means comprises a motor.

12. An MRI system for administering MRI to subjects, the MRI system comprising:

a) an MRI device for use in an MRI room;

b) a control room external to the MRI room;

c) a shielded interface unit in the MRI room for receiving a video input and an audio input and control signals from the control room, and for receiving subject generated sound input and subject generated control input;

e) a visual display for receiving from the interface unit the video input and for displaying to the subject in the MRI room visual images;

f) a sound suppression circuit in the interface unit for suppressing sound emanating from the MRI device by generating a sound suppression signal;

g) a sound transmission system for providing sound to the subject in the MRI room, wherein the sound transmission system receives the audio input and the sound suppression signal from the interface unit;

e) a microphone system in the MRI room for receiving subject generated sound for transmission to the interface unit as subject generated sound input;

f) a subject controllable input device in the MRI room for providing subject inputs to the interface unit; and

g) a subject monitor receiver in the interface unit for receiving physiological information about a subject.

1. A visual display for use by a subject in an MRI, the video display comprising left and right displays and distance adjustment means for adjusting the distance between the left and right displays, each display comprising:
a) an OLED for receiving the video input and transmitting video images; and
b) a prism receiving the video images from the OLED.

2. An MRI system for administering MRI to subjects, the MRI system comprising:
d) an MRI device for use in an MRI room;
e) a control room external to the MRI room;
f) a shielded interface unit in the MRI room for receiving a video input and an audio input and control signals from the control room, and for receiving subject generated sound input and subject generated control input;
g) a visual display for receiving from the interface unit the video input and for displaying to the subject in the MRI room visual images;
h) a sound suppression circuit in the interface unit for suppressing sound emanating from the MRI device by generating a sound suppression signal;
i) a sound transmission system for providing sound to the subject in the MRI room, wherein the sound transmission system receives the audio input and the sound suppression signal from the interface unit;
e) a microphone system in the MRI room for receiving subject generated sound for transmission to the interface unit as subject generated sound input;
f) a subject controllable input device in the MRI room for providing subject inputs to the interface unit; and
g) a subject monitor receiver in the interface unit for receiving physiological information about a subject.
